(19) United States
(12) Patent Application Publication
(10) Pub. No.: US 2017/0261493 A1
(43) Pub. Date: Sep. 14, 2017

(54) CELL CULTURE INTERFACES, SYSTEMS, AND USES THEREOF

(71) Applicant: Georgia Tech Research Corporation, Atlanta, GA (US)

(72) Inventors: MUHANNED S. BAKIR, ATLANTA, GA (US); HUA WANG, ATLANTA, GA (US); CHAOQI ZHANG, ATLANTA, GA (US)

(21) Appl. No.: 15/527,250

(22) PCT Filed: Nov. 17, 2015
(86) PCT No.: PCT/US15/61195

§ 371 Date: May 16, 2017

Related U.S. Application Data
(60) Provisional application No. 62/080,774, filed on Nov. 17, 2014.

(51) Int. Cl. G01N 33/50; C12M 1/36

(52) U.S. Cl. CPC G01N 33/502; C12M 41/48

(57) ABSTRACT

Described herein are cell culture interfaces that can be configured to relay an energy between a cell and an electronic interface and methods of using the cell culture interfaces that can be configured to relay an energy between a cell and an electronic interface.

### CROSS-REFERENCE TO RELATED APPLICATIONS

This application also claims the benefit of U.S. Provisional Application Ser. No. 62/080,774, filed on Nov. 17, 2014, the entirety of which is incorporated herein by reference.

### BACKGROUND

Cell culture systems remain a workhorse in both research and industry. Both research and industry demand high-throughput and low-cost systems. As such, there exists a need for the development of improved cell culture systems and devices.

### SUMMARY

Described herein are embodiments of a cell culture interface that can contain a first layer that can contain a via or a plurality of vias, where the via or plurality of vias can extend from a first surface of the first layer to a second surface of the first layer; and a second layer, where the second layer and/or the first layer can be optically transparent and can be configured to contact a cell culture, and where the cell culture interface can be configured to communicate and/or relay an energy between the cell culture and an electronic interface. In embodiments, each via can be independently selected from the group of: electrical vias and optical vias. In embodiments, at least one of the plurality of vias can be an electrical via and wherein the electrical via can further extend through the second layer from a first surface of the second layer to a second surface of the second layer. In embodiments, the first layer can be configured to removably couple to the electrical interface. In embodiments, the cell culture interface can be configured to optically, electrically, or optically and electrically communicate with and/or relay an energy between the electronic interface and cells cultured on the layered interface. In embodiments, the cell culture interface can contain a third layer. The third layer can be an optical filter. The third layer can couple to the second layer. In embodiments, the second layer is an optical filter. In embodiments the first and the second layers form a single layer, such that the first and the second layer are indistinguishable from each other. In embodiments, the first and the third layer form a single layer, such that the first and the third layer are indistinguishable from each other. In embodiments, the second and the third layer form a single layer. In embodiments, the first, second and third layer form a single layer such that the cell culture interface does not contain separately identifiable layers.

The first layer, second layer, and/or third layer can be optically transparent. The first layer, second layer, and/or third layer can be made of a polymer, glass, or polymer and glass. In some embodiments the polymer can be polypropylene, polystyrene, or PDMS. The cell culture interface can further include a structured layer that can be coupled to the second or optional third layer. The structured layer can contain a well and or/a microchannel. The structured layered can contain a plurality of wells and/or microchannels. The cell culture interface can be biocompatible and/or disposable.

Also provided herein are methods that include the step of culturing cells on a layered interface or system including the layered cell culture interface as described herein. In embodiments, the method can include the step of culturing cells on the layered interface. The method can include relaying an energy between the cells and an electronic interface through the layered interface. The method can further include the step of adding a compound to the cells or contacting a cell with a compound. The compound can be a biological molecule or a chemical compound. In embodiments, the cells can be cardiac cells, neurons, or embryonic stem cells.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 shows a cross sectional side view of embodiments of an electronic interface coupled to a substrate.

FIG. 2 shows a cross sectional side view of embodiments of a cell culture surface interface.

FIG. 3 shows embodiments of a system that can contain an electronic interface and a cell culture interface.

FIG. 4 shows embodiments of a system that can contain an electronic interface and a cell culture interface, where the cell culture interface can further contain a structured layer.

FIG. 5 shows embodiments of a system containing a plurality of electronic interfaces and a cell culture interface.

FIG. 6 shows embodiments of a cell culture interface configured as a single-well cell culture container.

FIG. 7 shows further embodiments of a cell culture interface configured as a multi-well cell culture container.

FIG. 8 shows further embodiments of a system that can contain an electronic interface and a cell culture interface where an interconnect is coupled to the electronic interface.

FIG. 9 shows further embodiments of a system that can contain an electronic interface and a cell culture interface where an interconnect can be coupled to the electronic interface and where an alignment structure can be coupled to the electronic interface.

FIG. 10 shows further embodiments of a system that can contain an electronic interface and a cell culture interface.

FIG. 11 shows further embodiments of a system that can contain an electronic interface, where an alignment structure can be coupled to the electronic interface.

FIG. 12 shows further embodiments of an electronic interface with an alignment structure configured to couple to an alignment structure on a substrate.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, cell biology, microbiology, nanotechnology, organic chemistry, biochemistry, chemical engineering, electrical engineering, computer engineering, biomedical engineering and the like, which are within the skill of the art. Such techniques are explained fully in the literature.

### DEFINITIONS

As used herein, “integrated circuit” can refer to a set of electronic, photonic, and/or opto-electronic circuits integrated on a semiconductor plate or wafer.

As used herein, “integrated circuit arrays” can refer to a set of integrated circuits where the integrated circuits are configured as a mappable, addressable, and/or ordered array of pixels or pixel groups.

As used herein “optically transparent” can refer to a property of a material that refers to the ability of the material to allow electromagnetic energy (e.g. light waves) to pass through. “Optically transparent” as used herein refers to any material that does not have 100% impendence of electromagnetic energy (e.g. light waves). An optically transparent material can allow 1% to 100% of all light waves or other electromagnetic energy to pass through.

As used herein, “about,” “approximately,” and the like, when used in connection with a numerical variable, generally refers to the value of the variable and to all values of the variable that are within the experimental error (e.g., within the 95% confidence interval for the mean) or within +/−10% of the indicated value, whichever is greater.

As used herein, “sensor” refers to a transducer that can convert a form of energy into an electrical, electromagnetic, or vibrational signal. Sensor(s), as used herein, can be configured to convert an energy input into an output signal, such as a voltage, impedance, sound, light, or other signal.

As used herein, “communication” refers to the transfer information and/or an energy signal between one or more devices or components thereof. Communication can be physical, electrical, electromagnetic, optical, mechanical, nuclear, atomic, visual, audible, molecular, thermal, fluidic, vibrational, wireless, chemical, and/or magnetic.

As used herein, “energy” can refer to any form of energy, including but not limited to, thermal energy, electromagnetic energy, vibrational energy, magnetic energy, chemical energy, electrical energy, mechanical energy, and elastic energy.

Discussion

Existing cell-based assays often rely on sensing a single modality (e.g. a fluorescent signal), which presents significant drawbacks such as expensive infrastructure, limited throughputs, and an inability for continuous and long-term monitoring. Therefore, there is a tremendous need for low-cost sensing platforms for cell-based assays to perform fast, efficient, and large-scale cell culture assays.

With that said, described herein are systems and devices that can be configured to relay an energy between a cell or cells of a cell culture and/or the cell culture environment and an electronic interface. Also described herein are methods of using the cell culture systems and devices provided herein. The systems and devices described herein can allow for low-cost, large-scale throughput, and real-time cellular monitoring. Further, the systems and devices described herein can be configured such that one or more components are disposable and/or reusable, which can reduce contamination concerns and decrease cost. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

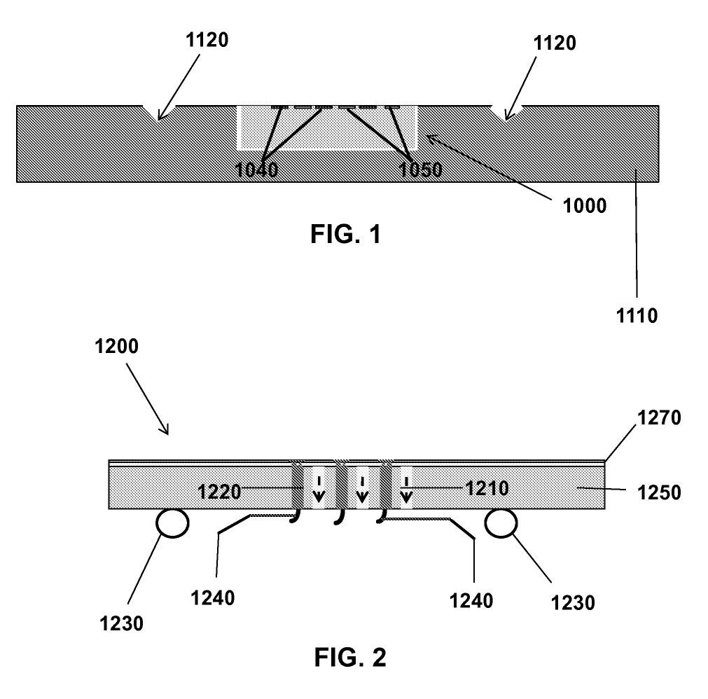

Described herein are systems that can contain cell culture interface, where the cell culture interface can be configured to contact a cell, cells, and/or cell culture and relay an energy between a cell or cells and an electronic interface. Discussion of the various embodiments begins with FIG. 1, which shows a side view of an electronic interface 1000, where the electronic interface can be coupled to a substrate 1110. The electronic interface can be embedded in the substrate 1110. The electronic interface 1000 can be attached to, attached on top of, or otherwise disposed on a surface of the substrate 1110 The electronic interface 1000 can be: selected from the group consisting of: a pixelated integrated circuit array, a complementary metal-oxide semiconductor microchip, microelectromechanical system, a silicon photonic chip, a charge-couple device, an application specific instruction set processor, an application specific integrated circuit, a complex programmable logic device, a field programmable gate array, a field programmable nanowire interconnect, a field programmable analog array, a field programmable object array, a generic array logic device, a macrocell array, a peripheral module interface, a programmable array logic, a programmable logic array, and a erasable programmable logic device. The electronic interface 1000 can include one or more electrical contacts 1040 and/or one or more optical receivers 1050. The electronic interface can include a printed circuit board. The electronic interface can contain a sensor. The electronic interface can include an actuator. The electronic interface 1000 can be coupled to and/or integrated with the substrate 1110. In some embodiments the electronic interface 1000 can be removably coupled to the substrate 1110. The substrate 1110 can be a base of support for the electronic interface 1000. The substrate 1110 can be made out of any desired material. Suitable materials include, but are not limited to, plastics, other polymers and polymer composites, rubbers, resins, ceramics, glass, silicon, and any combination thereof. In some embodiments, the substrate 1110 can be made entirely of or can includes polydimethylsiloxane (PDMS), plastic, glass, silicon, polypropylene, and/or polystyrene. some embodiments, the substrate can be or can include a printed circuit board. The substrate 1110 and/or electronic interface 1000 can also include an optical filter as a layer of the substrate and/or electronic interface similar to that described in reference to the cell culture interface (e.g. 1260, FIG. 2).

In some embodiments, the material can be a low cost disposable material, such as, but not limited to a plastic or glass. In some embodiments, the substrate 1110 and/or electronic interface 1000 can be configured for single use. In other embodiments, the substrate 1110 and/or electronic interface 1000 can be configured for multiple uses. In some embodiments, the substrate 1110 and/or electronic interface 1000 can be configured such that it or parts thereof can be cleaned and/or sterilized between uses. The substrate 1110 can also include one or more alignment structures 1120 that can be coupled to or integrated within the substrate 1110. The alignment structures 1120 and operation thereof are described in greater detail below.

Attention is now directed to FIG. 2, which shows embodiments of the cell culture interface 1200. The cell culture interface 1200 can be layered. The cell culture interface 1200 can contain a first layer 1250, where the first layer 1250 contains a via or a plurality of vias (e.g. 1210, 1220). The via or plurality of vias can extend from a first surface of the first layer to a second surface of the first layer 1250. Each via can each be independently selected from the group of electrical vias 1220 and optical vias 1210. Electrical vias can be made of an electrically conductive or semiconductive material. Suitable electrically conductive and semiconductive materials include but are not limited to platinum, gold, silver, copper, aluminum, and silicon. Optical vias 1210 can be made of an optically transparent material. The optically transparent material can be the same material or different material that the first layer 1250, second layer 1260 or the material of any additional layer. One or more of the optical vias can be or include an optical filter. One or more of the optical vias can be an optical fiber. The optical vias can be a hollow or mostly hollow structure such that the optical via is air. The optical via(s) can be made of the same or of a different material than a layer of the cell culture interface. The optical via(s) can be made out of plastic, other polymer, glass and the like.

The cell culture interface 1220 can further include a second layer 1260 and an optional third layer 1270. The second layer can be coupled to the first layer 1250. The optional third layer 1270 can be coupled to the first layer 1250. The second layer 1260 can be optically transparent. The third layer 1270 can be optically transparent. The second layer 1260 can be an optical filter. The third layer can be an optical filter. Where both a second layer 1260 and third layer 1270 are included and both are configured as optical filters, each layer can be the same optical filter or different optical filters. The second layer 1260 can be configured to contact a cell or cell culture. The third layer 1270 can be configured to contact a cell or cell culture. In some embodiments, the cell culture interface 1200 can be configured such that the second layer is disposed between the third layer 1270 and the first layer 1250. In other embodiments, the cell culture interface can be configured such that the third layer 1270 is sandwiched in disposed between the second layer 1250 and the first layer 1250. The cell culture interface 1200 can include one or more additional layers. The first, second, and optional third layer can be made of the same material and made at the same time such that they form substantially a single layer.

The cell culture interface 1200 can be configured to relay an energy between a cell and the electronic interface 1000. This relay can occur through the via(s). Where the via is an electrical via 1220, the via can extend through the second layer 1260, optional third layer 1270, and any other layer present in the cell culture interface 1200 that is between the first layer and the cell culture. The extension of the electronic via through the second layer 1260 and/or optional third layer 1270 can be flexible or inflexible wire bonds.

Any layer or component of the cell culture interface 1200 can be made out of any desired material. Suitable materials include, but are not limited to, plastics, other polymers and polymer composites, rubbers, resins, ceramics, glass, and any combination thereof. In some embodiments, the substrate includes polydimethylsiloxane (PDMS), polypropylene, and/or polystyrene. The layers can be made of the same material or different materials. In some embodiments, the material can be a low cost disposable material such as a plastic or glass. In some embodiments, the cell culture interface 1200 can be configured for single use. In other embodiments, the cell culture interface 1200 can be configured for multiple uses. In some embodiments, the cell culture interface 1200 can be configured such that it or parts thereof can be cleaned and/or sterilized between uses.

The layer that is configured to contact a cell and/or cell culture can be treated. As used herein the term “treated” refers to a modification of a surface that is used for cell culture that facilitates attachment and/or growth of the cell or cells in the cell culture. Exemplary modifications include, but are not limited to, poly-lysine and other treatments that render the surface hydrophilic. In some embodiments, the layer that is configured to contact a cell and/or cell culture can be coated with a natural or synthetic matrix (e.g. cellular matrix), cellular matrix component, fibronectin, blocking reagent, antibody, and/or other molecule (e.g. strepavidin) as desired. Other suitable coatings will be appreciated by those of skill in the art.

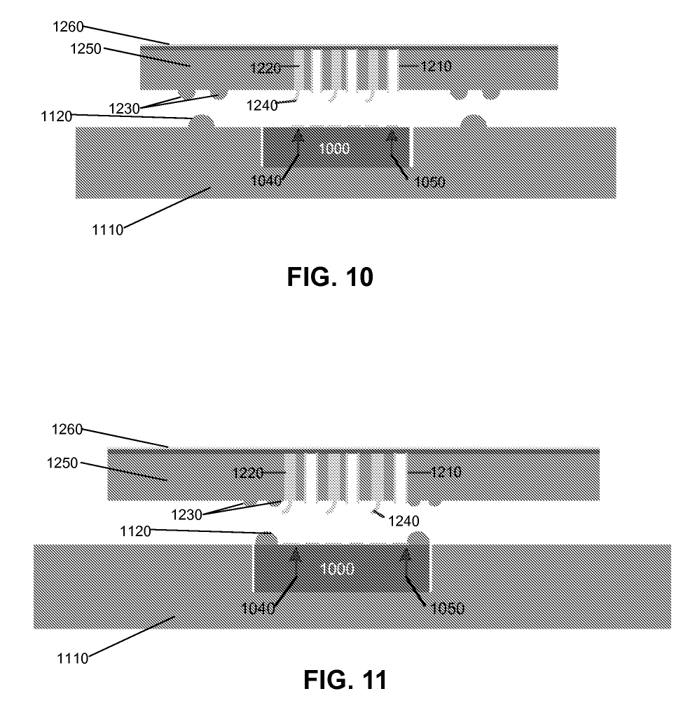

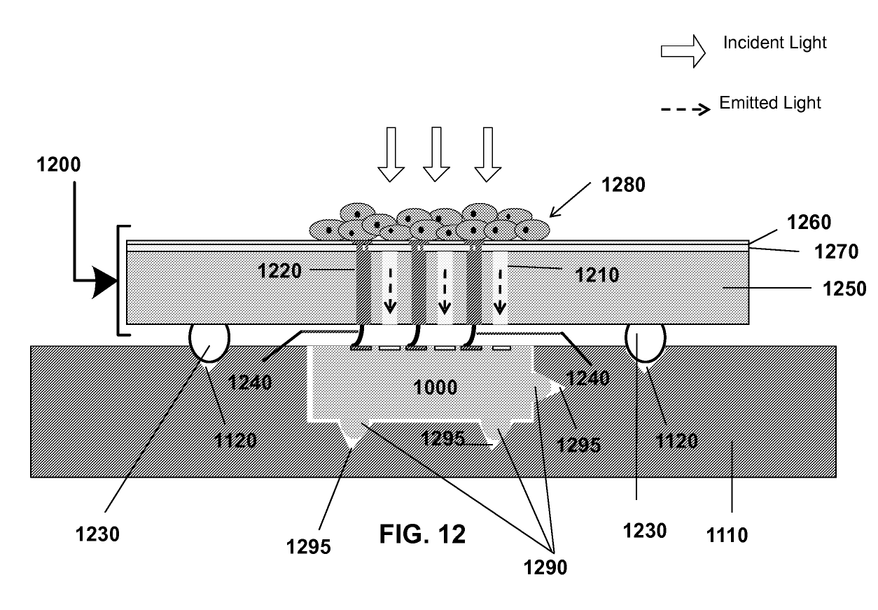

The cell culture interface 1200 can further include one or more interconnects 1240. See also FIGS. 19 and 20. In some embodiments, the interconnect(s) can be coupled to an electrical via 1220 of the cell culture interface 1200 and be configured to removably couple to an electrical contact 1040. See e.g. FIGS. 11, 19, and 20. In some embodiments, interconnect(s) can be coupled to the electrical contact(s) 1040 and configured to removably coupled to the electrical via(s) 1220. See e.g. FIGS. 17 and 18. The interconnect(s) can be mechanical interconnects. The interconnect(s) can be flexible interconnects such that each interconnect can deform to create a reliable electrical interface. The interconnects can be configured to couple an electronic via 1220 to an electrical contact 1040 of the electronic interface 1000. The interconnects can be made out of or include any suitable material. The material can be a conductive or semiconductive material. Suitable electrically conductive and semiconductive materials include but are not limited to platinum, gold, silver, copper, aluminum, and silicon and combinations thereof. The interconnects 1240 can be formed using microfabrication technology including lithography and electroplating; wirebonding methods in which free standing material is created; note these interconnects can be embedded within the manufacturing process of the removal/disposable layer, which can include injection molding, additive manufacturing, etc.

The cell culture interface 1200 can further contain one or more alignment structures 1230. The alignment structures 1230 can be coupled to or integrated within the first layer 1250. The alignment structures 1230 and their operation are discussed in further detail below.

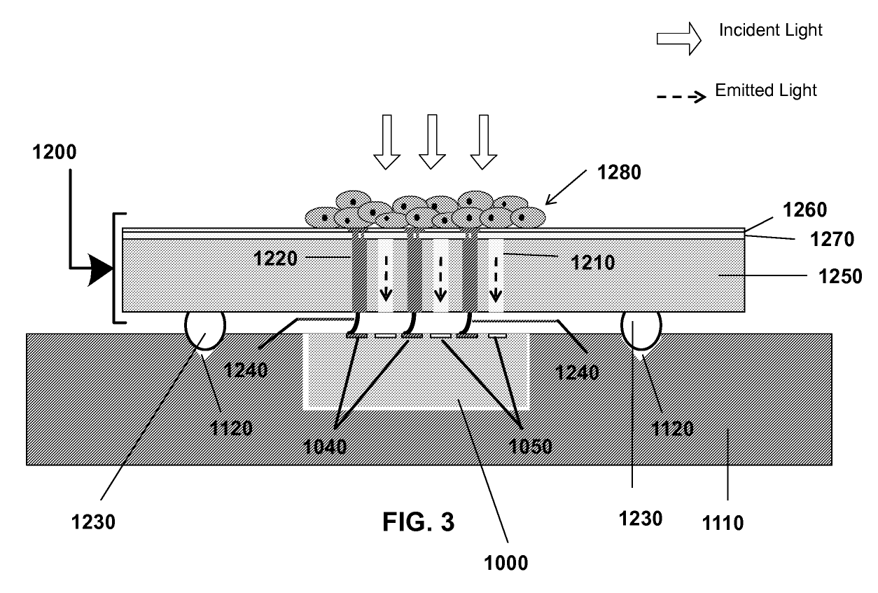

With the description of embodiments of the cell culture interface 1200 in mind attention is directed to FIG. 3, which shows embodiments of a system that can contain an electronic interface 1000 and a cell culture interface 1200. The electronic interface 1000 can be optically and/or electrically coupled to with the cell culture interface 1200 by a via or plurality of vias 1210, 1220. As described in greater detail below, the electronic interface can be incorporated into a substrate 1110. The substrate 1110 can be mechanically coupled to the cell culture interface 1200 through coupling of one or more alignment structures 1230, 1120. The electronic interface can be electrically and/or optically coupled with the cell culture interface 1200. The electronic interface 1000 can be mechanically and/or electrically coupled to the cell culture interface 1200 through the mechanical interconnect(s) 1240 that can extend between an electrical contact 1040 and an electrical via 1220. The substrate 1110 and/or the electrical interface 1000 can be removably coupled to the cell culture interface 1200. The layered interface 1200 can be configured to relay an energy between cell 1280 and electrical interface 1000 through a via 1210, 1220. It will be appreciated that energy relay can occur from the cells 1280 to the electronic interface 1000 or from the electronic interface 1000 to the cell(s) 1280.

In operation, cells can be cultured on the top surface of the cell culture interface 1200. An energy from a cells, cells, cell culture, or cell culture environment can be electrically and/or optically relayed to the electronic interface 1000 through the electronic and/or optical vias 1220, 1210 of the cell culture interface. A sensor in the electronic interface 1000 can detect the electrical or optical energy relayed from the cell by the cell culture interface 1200.

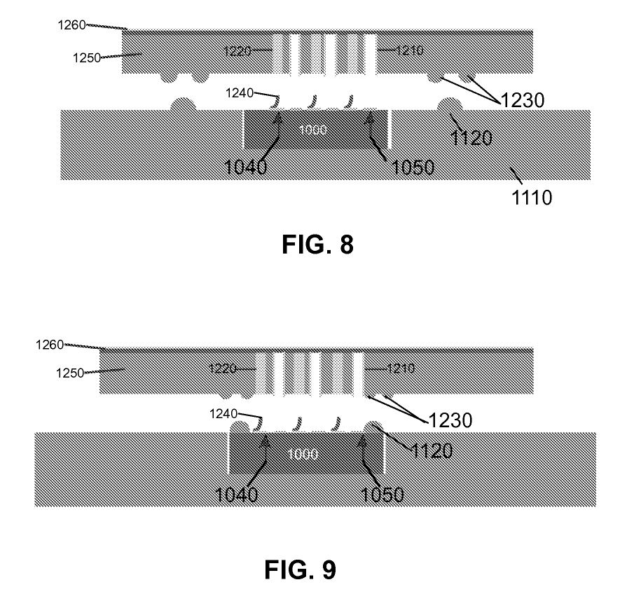

As shown in e.g. FIGS. 1-3, the substrate 1110 and the first layer 1250 of the layered interface can include one or more alignment structures 1120 and 1230. The alignment structures can be configured as a male or female structure that can be coupled to an alignment structure of the opposite configuration. For example, as shown in FIG. 1, the alignment structure can be configured as a female alignment structure 1120 that can couple with a male alignment structure on the cell culture interface 1200. The alignment structures can have any desired shape or size. In other embodiments, the alignment structures can be magnets. The alignment structure(s) 1120 of the substrate 1110 can have one polarity (either N or S) and the alignment structure(s) 1230 of the cell culture interface 1200 can have the opposite polarity. In some embodiments, such as those illustrated in FIG. 3, the alignment structure can be a semispehere (e.g. 1230) or an inverted pit (e.g. 1120). The inverted pit can be a pyramid, cube, cone, or other shape. Other configurations of the alignment structures are shown in FIGS. 8-11. The alignment structures can be made by microfabrication, injection modeling, additive manufacturing, and the like. Other suitable methods of manufacture are described elsewhere herein.

The alignment structures can be located on any surface of the substrate 1110, electronic interface 1000, and/or the cell culture interface 1200. In embodiments, the substrate 1110 and the cell culture interface 1200 such that when the alignment structure(s) 1120 of the substrate are coupled to the alignment structure(s) 1230 of the cell culture interface 1200, the via(s) of the cell culture interface 1200 and any mechanical interconnects align with and/or electrically and/or optically couple with optical receiver 1050 and/or electrical contact(s) 1040 of the electronic interface 1000. In this way, the electronic interface 1000 can be easily self-aligned with the cell culture interface 1200. Further the alignment structures can be configured to allow the cell culture interface 1200 to removably couple to the electronic interface 1000 and/or substrate 1110.

In some embodiments, as shown in FIG. 12, a surface of the electronic interface 1000 can have one or more alignment structures 1290 that are configured to couple to one or more alignment structures on the substrate 1295 that are separate from an alignment structure on the substrate that is configured to couple to an alignment structure on the cell culture interface. These alignment structures 1290,1295 can align the electronic interface on or within the substrate independent of any alignment with the cell culture interface.

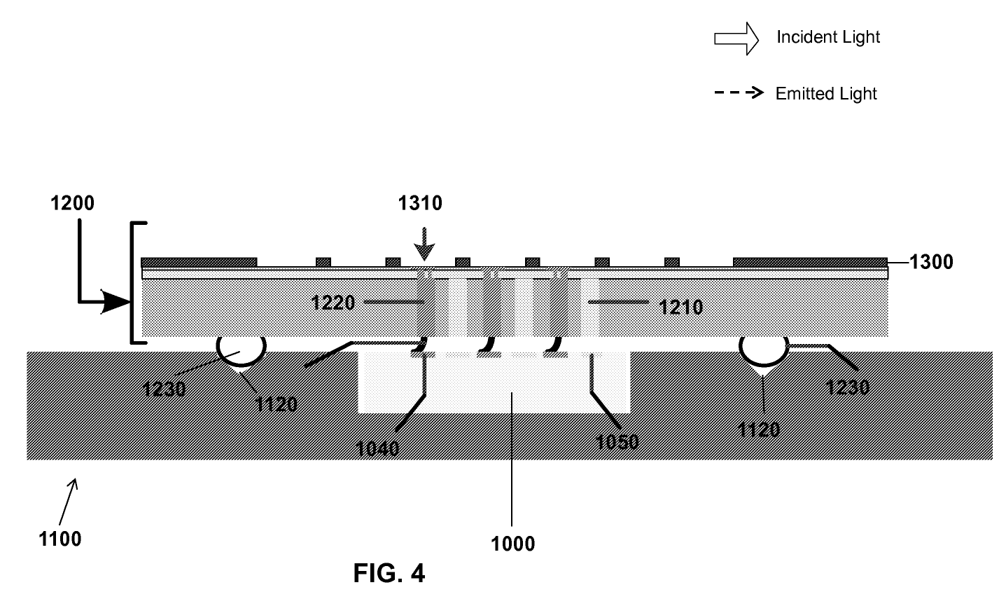

Attention is now turned to FIG. 4, which illustrates other embodiments of the system containing an electronic interface 1000 and a cell culture interface 1200, where the cell culture interface 1200 can further contain a structured layer 1300. The structured layer 1300 can contain one or more wells and/or channels 1310. The well(s) and/or channel(s) can be configured to contain one or more cells. The channels can be microchannels. The channels can be in fluidic communication with each other or one or more wells. The channels can be configured to allow fluid flow in and out of one or more wells. The walls of the well(s) and/or channel(s) can be hydrophobic. The walls of the well(s) and/or channel(s) can be treated and/or coated.

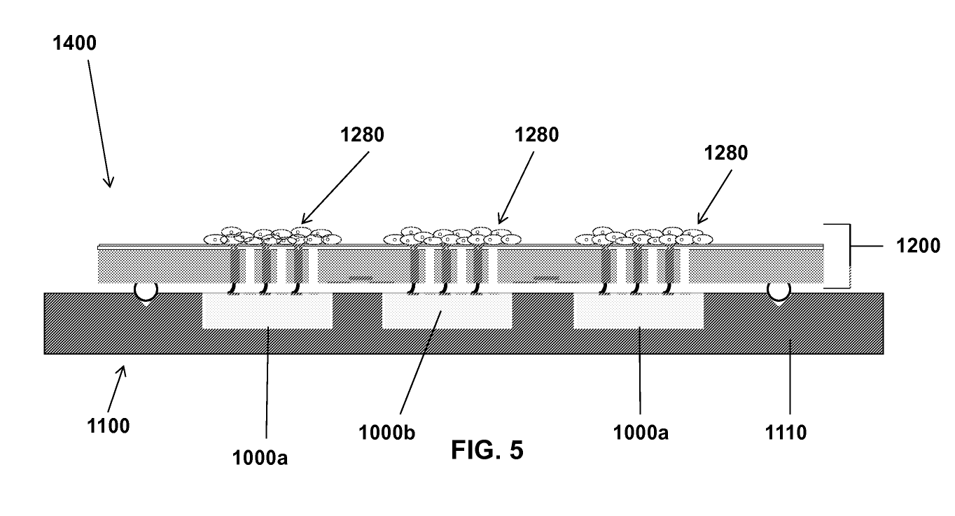

FIG. 5 illustrates other embodiments of a system 1400 containing a plurality electrical interfaces 1000a-c and a cell culture interface 1200. Each of the electrical interfaces 1000a-c of can be the same or different from one other of the electrical interfaces 1000a-c. It will be appreciated that the system can contain any number of cell culture interfaces 1000a . . . n, where n indicates any number. The number of electrical interfaces can range from 1 to 200 or more. In embodiments, each electrical interface can be independently selected from the group consisting of: a pixelated integrated circuit array, a complementary metal-oxide semiconductor microchip, microelectromechanical system, a silicon photonic chip, a charge-couple device, an application specific instruction set processor, an application specific integrated circuit, a complex programmable logic device, a field programmable gate array, a field programmable nanowire interconnect, a field programmable analog array, a field programmable object array, a generic array logic device, a macrocell array, a peripheral module interface, a programmable array logic, a programmable logic array, and a erasable programmable logic device. The cell culture interface 1200 in the system 1400 can, in some embodiments, further contain a structured layer 1300. The structured layer can be configured such that different wells or channels confine different groups of cells to one electrical interface (e.g. 1000 a, b, or c) of the plurality electrical interfaces 1100a-c. In other embodiments, the structured layer can be configured such that different wells or channels confine different groups of cells to a subset (e.g. 1000 a and b, 1000 a and c, or 1000 b and c) of the plurality of electrical interfaces 1000a-c.

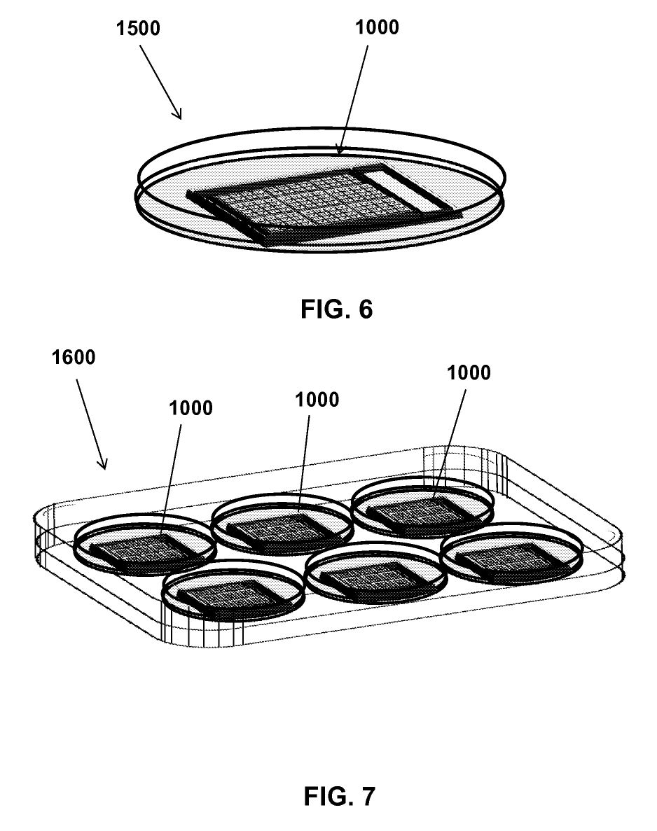

As is illustrated in FIGS. 6-7, the electrical interface 1000 and cell culture interface 1200 can be incorporated into or configured as cell culture containers, such as single-well plates (e.g. FIG. 6), multi-well plates (FIG. 7), and cell culture-flasks. In other words, the cell culture interface 1200 can be configured as a cell culture container, such as a single-well plate (e.g. FIG. 6), multi-well plates (FIG. 7) and cell culture flasks. Other suitable containers configuration will be appreciated by those of skill in the art.

The devices and components thereof described herein can be manufactured by any suitable method and in any suitable way. Suitable methods include, but are not limited to, injection molding, 3-D printing, glass/plastic molding processes, optical fiber production process, casting, chemical deposition, electrospinning, machining, die casting, evaporative-pattern casting, resin casting, sand casting, shell molding, vacuum molding, thermoforming, laminating, dip molding, embossing, drawing, stamping, electroforming, laser cutting, welding, soldering, sintering, bonding, composite material winding, direct metal laser sintering, fused deposition molding, and stereolithography. Other techniques will be appreciated by those of skill in the art.

We claim:

1. A system comprising:
an electronic interface;
a substrate, where the substrate is coupled to the electronic interface; and
a cell culture interface comprising:
a first layer comprising a via, where the via extends from a first surface of the first layer to a second surface of the first layer;
a second layer, where the second layer is optically transparent and is configured to contact a cell culture; and
where the cell culture interface is configured to relay an energy between a cell and the electronic interface.

2. The system of claim 1, wherein the electronic interface is selected from the group consisting of: a pixelated integrated circuit array, a complementary metal-oxide semiconductor microchip, microelectromechanical system, a silicon photonic chip, a charge-couple device, an application specific instruction set processor, an application specific integrated circuit, a complex programmable logic device, a field programmable gate array, a field programmable nanowire interconnect, a field programmable analog array, a field programmable object array, a generic array logic device, a macrocell array, a peripheral module interface, a programmable array logic, a programmable logic array, and a erasable programmable logic device.

3. The system of claim 1, wherein the via is selected from the group consisting of: electrical vias and optical vias.

4. The system of claim 1, further comprising a plurality of vias, where in each via of the plurality of vias is independently select from the group consisting of: electrical vias and optical vias.

5. The system of claim 1, wherein the substrate is removably coupled to the cell culture interface.

6. The system of claim 1, wherein the electronic interface is configured to electrically, optically, or electrically and optically couple to the cell culture interface.

7. The system of claim 1, wherein the first layer and the second layer are substantially the same.

8. The system of claim 1, wherein the first layer and the second layer are substantially different.

9. The system of claim 1, wherein the cell culture interface is biocompatible.

10. The system of claim 1, wherein the cell culture interface further comprises an alignment structure.

11. The system of claim 10, wherein the substrate further comprises an alignment structure.

12. The system of claim 10, wherein the cell culture interface is coupled to the substrate via the alignment structures.

13. The system of claim 1, wherein the layered interface comprises a third layer, wherein the third layer is an optical filter and wherein the third layer is coupled to the second layer.

14. The system of claim 1, wherein the substrate comprises a polymer or glass.

15. The system of claim 1, wherein the first layer, the second layer, or both the first and the second layer comprise a polymer or glass.

16. The system of claim 1, wherein the layered interface further comprises a structured layer and wherein the structured layer is coupled to the second layer.

17. The system of claim 16, wherein the structured layer comprises a well, a microchannel, or both a well and a microchannel.

18. A cell culture interface comprising:
a first layer comprising a via, where the via extends from a first surface of the first layer to a second surface of the first layer; and
a second layer, where the second layer is optically transparent and is configured to contact a cell culture,
where the cell culture interface is configured to relay an energy between a cell and an electronic interface.

19. The cell culture interface of claim 18, wherein the via is selected from the group consisting of: electrical vias and optical vias.

20. The cell culture interface of claim 18, further comprising a plurality of vias, where in each via of the plurality of vias is independently select from the group consisting of: electrical vias and optical vias.

21. The cell culture interface of claim 20, wherein at least one of the plurality of vias is an electrical via and wherein the electrical via further extends through the second layer from a first surface of the second layer to a second surface of the second layer.

22. The cell culture interface of claim 18 wherein the via is an electrical via and wherein the electrical via further extends through the second layer from a first surface of the second layer to a second surface of the second layer.

23. The cell culture interface of claim 18, wherein the first layer is configured to removably couple to an electronic interface or a substrate, where the substrate is coupled to an electronic interface.

24. The cell culture interface of claim 18, wherein the first layer and the second layer are substantially the same.

25. The cell culture interface of claim 18, wherein the first layer and the second layer are substantially different.

26. The cell culture interface of claim 18, wherein the cell culture interface comprises an alignment structure, where the alignment structure is coupled to or integrated within the first layer.

27. The cell culture interface of claim 18, wherein the cell culture interface is configured to optically, electrically, or optically and electrically relay an energy between a cell and an electronic interface.

28. The cell culture interface of claim 18, wherein the first layer, the second layer, or both the first and the second layer comprise a polymer or glass.

29. The cell culture interface of claim 18, wherein the cell culture interface is biocompatible.

30. The cell culture interface of claim 18, further comprising a structured layer, wherein the structured layer is coupled to the second layer.

31. The cell culture interface of claim 30, wherein the structured layer comprises a well, a microchannel, or both a well and a microchannel.

32. A method comprising:
culturing cells on a layered interface; and
relaying an energy between a cell and an electronic interface through a via of the layered interface.

33. The method of claim 32, wherein the cells are embryonic stem cells, neurons, or cardiac cells.

34. The method of claim 32, further comprising the step of adding a compound to the cells.
